(19) United States
(12) Patent Application Publication
(10) Pub. No.: US 2025/0076417 A1
(43) Pub. Date: Mar. 6, 2025

(54) SENSOR LAYOUT IN A MAGNETOMETER SYSTEM

(71) Applicant: FieldLine Inc., Boulder, CO (US)

(72) Inventors: Svenja Knappe, Boulder, CO (US); Orang Alem, Erie, CO (US); Kenneth J. Hughes, Lafayette, CO (US)

(21) Appl. No.: 18/826,810

(22) Filed: Sep. 6, 2024

Related U.S. Application Data
(60) Provisional application No. 63/580,734, filed on Sep. 6, 2023.

(51) Int. Cl. G01R 33/00; A61B 5/00; A61B 5/245; G01R 33/02; G01R 33/022; G01R 33/032; G01R 33/035

(52) U.S. Cl. CPC G01R 33/0094; G01R 33/0047; G01R 33/0206; A61B 5/245; A61B 5/6803; A61B 5/6805; G01R 33/0213; G01R 33/022; G01R 33/032; G01R 33/0354

(57) ABSTRACT

Various embodiments disclosed herein comprise systems and methods to arrange magnetic field sensors. In some examples a magnetic field detection system comprises a sensor holder and magnetometers. The sensor holder mounts the magnetometers proximate to a magnetic field source that generates a magnetic field. The magnetometers measure the magnetic field in multiple directions. A first portion of the magnetometers measures a normal component of the magnetic field and a first tangential component of the magnetic field, and a second portion of the magnetometers measures the normal component of the magnetic field and a second tangential component of the magnetic field. The sensor holder distributes the first portion of the magnetometers and the second portion of the magnetometers, so the tangential components measured by neighboring ones of the magnetometers mounted to the sensor holder are orthogonal.

### RELATED APPLICATIONS

This Patent application claims the benefit of and priority to U.S. Provisional Patent Application 63/580,734 titled, “SENSOR LAYOUT IN A MAGNETOMETER SYSTEM” which was filed on Sep. 6, 2023, and which is hereby incorporated by reference in its entirety into this Patent Application.

### TECHNICAL FIELD

Various embodiments of the present technology relate to magnetic sensing and more specifically, to anatomical magnetic sensor layouts.

### BACKGROUND

Magnetometer systems detect and characterize magnetic fields generated by a magnetic field source. The magnetometer systems measure the field strength and/or direction of the magnetic fields to characterize the sensed fields. Magnetoencephalography (MEG) systems are a type of magnetometer system that measures magnetic fields generated by neuronal activity within a subject's brain to map brain function. MEG systems image brain activity by detecting magnetic fields from neural currents using an array of magnetic sensors placed near the head of a subject and then computing the locations of the neural activity relative to the location of the sensor in a process referred to as source localization. Exemplary magnetic sensors used in the MEG systems include Optically Pumped Magnetometers (OPMs), however other magnetometer types like Superconducting Quantum Interference Devices (SQUIDs) may be used. The data from the sensors along with each sensor location is used to calculate the locations of neuronal signal sources to form MEG images of brain activity. Some MEG systems have sensors that can move independently and conform to the size and shape of the head. These MEG systems are referred to as on-scalp or conformal MEG.

In conformal MEG, the magnetometers are mounted to a sensor holder and the sensor holder is worn by a target. Exemplary sensor holders for MEG include helmets and flexible caps. The sensor holder contours the magnetometers to the target. The magnetometers detect and characterize the target's magnetic field in response to power and signaling received from a controller. The magnetometers transfer signals that characterize the sensed magnetic field over cables to the controller. The controller processes the signals to model the target magnetic field. Each magnetometer measures one, two, or three orthogonal components of the target magnetic field at the location of the magnetometer. The distribution of components determines the quality of reconstruction of the target magnetic fields. Although three-axis magnetometers may be used in all locations on the sensor mount, this results in an inefficient allocation of resources as the number of tangential measurement components is excessive. Moreover, three-axis magnetometers are expensive. Unfortunately, magnetometer systems do not effectively or efficiently allocate measurement resources.

### Overview

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to solutions for measuring magnetic fields. Some embodiments comprise a magnetic field detection system to arrange magnetometers. The magnetic field measurement system comprises a sensor holder and magnetic field sensors. The sensor holder mounts the magnetometers proximate to a magnetic field source that generates a magnetic field. The magnetometers measure the magnetic field in multiple directions. A first portion of the magnetometers measures a normal component of the magnetic field and a first tangential component of the magnetic field. A second portion of the magnetometers measures the normal component of the magnetic field and a second tangential component of the magnetic field. The sensor holder distributes the first portion of the magnetometers and the second portion of the magnetometers so that the tangential components measured by neighboring ones of the magnetometers mounted to the sensor holder are different.

Some embodiments comprise a method to arrange magnetometers. The method comprises mounting magnetometers on a sensor holder. The method further comprises distributing the magnetometers on the sensor holder so tangential field components measured by adjacent ones of the magnetometers are different. The method further comprises placing the sensor holder on a magnetic field source that generates a magnetic field to position the magnetometers proximate to the magnetic field source. The method further comprises utilizing a first portion of the magnetometers to measure a normal component of the magnetic field and a first tangential component of the magnetic field. The method further comprises utilizing a second portion of the magnetometers to measure the normal component of the magnetic field and a second tangential component of the magnetic field.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary magnetic field detection system.

FIG. 2A illustrates an exemplary magnetometer arrangement in the magnetic field detection system.

FIG. 2B illustrates a first exemplary magnetometer arrangement in the magnetic field detection system.

FIG. 3 illustrates a second exemplary operation of the magnetic field detection system.

FIG. 4 illustrates an exemplary Magnetoencephalography (MEG) system.

FIG. 5 illustrates exemplary Optically Pumped Magnetometer (OPM) measurement channels in the MEG system.

FIG. 6 further illustrates the exemplary OPM measurement channels in the MEG system.

FIG. 7 illustrates a first exemplary OPM arrangement in the MEG system.

FIG. 8 illustrates a MEG helmet implementing the first exemplary OPM arrangement in the MEG system.

FIG. 9 illustrates a second exemplary OPM arrangement in the MEG system.

FIG. 10 illustrates the MEG helmet implementing the second exemplary OPM arrangement in the MEG system.

FIG. 11 illustrates an exemplary OPM in the MEG system.

FIG. 12 illustrates an exemplary MEG controller in the MEG system.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### Technical Description

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

The examples presented herein comprise systems and methods to optimize sensor layout in magnetic field detection systems. In conventional Magnetoencephalography (MEG), a set of magnetic field sensors is arranged on a helmet or other suitable headgear and placed on the head of the subject. The sensors measure all three orthogonal components (i.e., the normal component and both tangential components of the magnetic field with respect to the surface of the subject) of the subject's magnetic field at all locations on the MEG headgear. While all field components may be used to reconstruct the target magnetic field sources inside the head, in most cases, the normal components of the magnetic field are used for case and accuracy of reconstructions. The tangential field components measured by the sensors are used to reconstruct and reject ambient magnetic field signals. Since the target magnetic field sources inside the subject are much closer than most ambient sources, using two tangential measurement channels at all sensor locations is wasteful. Therefore, conventional MEG systems do not efficiently allocate resources.

To overcome the above-described problems in conventional MEG systems, a sensor layout for a magnetometer system is presented herein. The magnetometer system comprises sensors that measure two orthogonal (or near orthogonal) magnetic field components. The sensors are mounted to a sensor holder. The sensors are arranged on the holder so that all sensors measure the normal component (with respect to a target surface) of the magnetic field and one tangential component (with respect to a target surface) of the magnetic field. The tangential components measured by neighboring sensors on the sensor holder are orthogonal or near orthogonal. It is beneficial to sample all field components around the head of the subject to detect both the target field and ambient noise. The target magnetic field sources inside the head are typically stronger than ambient sources. To optimize resources, the sensor layout utilizes a higher density of sensors measuring the normal field components and a lower density of sensors measuring each of the tangential components to measure the ambient magnetic noise sources. The sensor layout distributes the tangential components in the two orthogonal directions equally (or approximately equal) to avoid biasing reconstruction solutions. The biggest contributions to ambient “noise” signals are movement-related artifacts. While these artifact signals are big, the spatial wavelength is usually also large. Therefore, using half the channels for the suppression of these artifacts is sufficient which leaves the other half of the channels in the normal direction to sample the signals of interest from the target magnetic field with high fidelity. Now referring to the Figures.

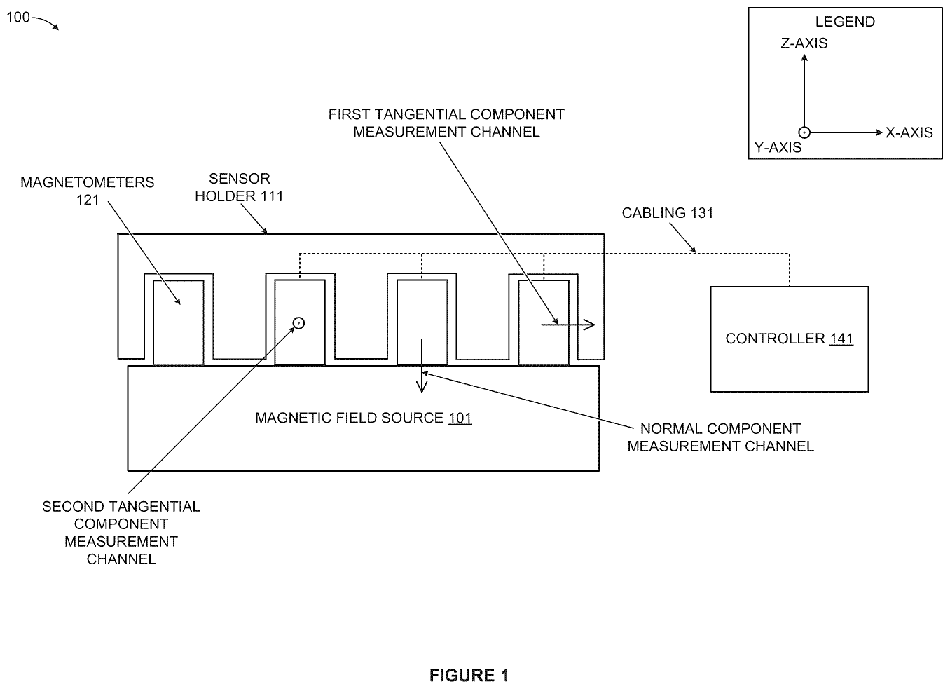

FIG. 1 illustrates magnetic field detection system 100 to arrange magnetometers. Magnetic field detection system 100 performs operations like detecting magnetic fields and relating the detected magnetic fields to a magnetic field source. For example, magnetic field detection system may be representative of an MEG system, Magnetocardiography (MCG) system, Magnetogastrography (MGG) system, Magnetomyography (MMG) system, Magnetoneurography (MNG) system, and/or another type of magnetic field detection system. System 100 comprises magnetic field source 101, sensor holder 111, magnetometers 121, cabling 131, and controller 141. Magnetic field source 101 generates a target magnetic field. Magnetic field source 101 may comprise any magnetic field source including non-biological magnetic field sources. In other examples, magnetic field detection system 100 may differ. System 100 may include fewer or additional components than those illustrated in FIG. 1. Likewise, the illustrated components of system 100 may include fewer or additional components, assets, or connections than shown.

Various examples of magnetic field detection system operation and configuration are described herein. In some examples, sensor holder 111 mounts magnetometers 121 proximate to magnetic field source 101. Magnetic field source 101 generates a magnetic field. Magnetometers 121 measure the magnetic field in multiple directions. A first portion of magnetometers 121 measures a normal component of the magnetic field (i.e., normal to the surface of magnetic field source 101) and a first tangential component of the magnetic field (i.e., not normal to the surface of magnetic field source 101. A second portion of magnetometers 121 measures the normal component of the magnetic field and a second tangential component of the magnetic field. Magnetic field components running in line with the page are illustrated as arrows while magnetic field components running through the page are illustrated as circles. Sensor holder 111 distributes the first portion of magnetometers 121 and the second portion of the magnetometers 121 so that the tangential components measured by neighboring ones of magnetometers 121 mounted to sensor holder 111 are different. For example, one of magnetometers 121 in a first slot of sensor holder 111 may measure the normal component and the first tangential component of the magnetic field while another one of magnetometers 121 in a neighboring slot of sensor holder 111 may measure the normal component and the second tangential component of the magnetic field.

Sensor holder 111 comprises an apparatus to mount magnetometers 121. Holder 111 may comprise a rigid helmet, a flexible hat, a blanket (e.g., a flexible sensor holder), a sleeve, a vest, and the like. Typically, holder 111 is wearable by magnetic field source 101. For example, if magnetic field source 101 comprises an adult human, holder 111 may be shaped to fit over part of the human body (e.g., the head, chest, arm, leg, etc.), however the shape of sensor holder 111 nor the target type is not limited. Sensor holder 111 may be representative of a sensor holder to perform MEG, MCG, MGG, MMG, MNG, or another type of anatomical magnetic field sensing technology. Holder 111 may be constructed from plastic, carbon fiber, polymer, rubber, fabric, canvas, or other materials that provide structural support to holder 111 and that do not interfere in the sensing operations of magnetometers 121. As illustrated in FIG. 1, holder 111 comprises slots for magnetometers 121. Magnetometers 121 fit into the slots to connect to holder 111. Magnetometers 121 may connect to holder 111 using clamps, male/female sockets, ratchet mechanisms, set screws, springs, pistons, pneumatics, electronic actuators, and the like. The connectors between magnetometers 121 and holder 111 may be adjustable or static. For example, when holder 111 is worn by magnetic field source 101, the connectors may propel magnetometers 121 through their respective slots until in contact with magnetic field source 101 to contour magnetometers 121 to the surface geometry of magnetic field source 101. Holder 111 may comprise embedded circuitry to communicatively couple magnetometers 121 and controller 141 via cabling 131. Alternatively, holder 111 may comprise conduit (or another type of passage) for cabling 131 to communicatively couple magnetometers 121 and controller 141.

Magnetometers 121 measure magnetic fields generated by magnetic field source 101. For example, when magnetic field source 101 comprises a human being, magnetometers 121 may sense magnetic fields generated in the brain, heart, muscles, and the like in magnetic field source 101. Magnetometers 121 may be used to perform MEG, MCG, MGG, MMG, MNG, and/or other types of anatomical magnetic field sensing operations. Magnetometers 121 may comprise atomic magnetometers, Optically Pumped Magnetometers (OPMs), gradiometers, nitrogen vacancy centers, Superconducting Quantum Interference Devices (SQUIDs), magneto-resistive sensors, and/or other types of magnetic sensing devices. Magnetometer 121 may comprise subcomponents like atomic vapor cells, lasers, heaters, coils, photodetectors, processing circuitry, flash circuitry, and communication circuitry.

Magnetometers 121 are directional and may measure magnetic fields along one or two measurement axes. FIG. 1 includes a legend that defines an x, y, and z-axis. The x-axis runs along the horizontal of the page, and z-axis runs along the vertical of the page, and the y-axis rungs through the page. As illustrated in FIG. 1, the measurement axes of magnetometers 121 comprise a normal component measurement channel, a first tangential component measurement channel, and a second tangential component measurement channel. The normal component channel is parallel with the z-axis and detects magnetic field components normal to the surface of magnetic field source 101. The first tangential component measurement channel is parallel with the x-axis and detects magnetic field components that are not normal to the surface of magnetic field source 101. The second tangential component measurement channel is parallel with the y-axis and detects magnetic field components that are not normal to the surface of magnetic field source 101. The first and second tangential measurement channels may be orthogonal with respect to each other and with the normal measurement channel. Alternatively, the first and second tangential measurement channels may be near orthogonal (e.g., equal to or less than 10 degrees of orthogonal) with respect to each other and with the normal measurement channel. Magnetometers 121 may comprise one or two measurement channels. When magnetometers 121 comprise two measurement channels, each of magnetometers 121 comprises a normal measurement channel and one of the tangential component measurement channels to maintain the normal channel as the dominant sensing mode. When magnetometers 121 comprise a single measurement channel, a plurality, half, or a majority of magnetometers 121 comprises a normal measurement channel while the remining ones of magnetometers alternate between the two tangential component measurement channels.

Controller 141 comprises one or more computing devices that control the operation of magnetometers 121 to sense magnetic fields generated by magnetic field source 101. Controller 141 is communicatively coupled to magnetometers 121 over cabling 131. The communication links may comprise metallic links, glass fibers, radio channels, or some other communication media. The links may use inter-processor communication, bus interfaces, Ethernet, WiFi, virtual switching, and/or some other communication protocol. For example, the communication links may be supported by sheathed metallic wires. Alternatively, the communication links may be supported by a wireless transceiver (e.g., antennas) to exchange signaling between controller 141 and magnetometers 121 over a wireless networking protocol like Bluetooth. Controller 141 may supply electric current over a wired connection to power magnetometers 121. Alternatively, magnetometers 121 may be battery powered.

The one or more computing devices of controller 141 comprise processors, memory, lock-in amplifiers, modulators, and transceivers that are connected over bus circuitry. The processors may comprise Central Processing Units (CPUs), Graphical Processing Units (GPUs), Digital Signal Processors (DSPs), Application Specific Integrated Circuits (ASICs), Field Programmable Gate Arrays (FPGAs), and the like. The lock-in amplifiers comprise demodulators and low-pass filters. The memory may comprise Random Access Memory (RAM), flash circuitry, Solid States Drives (SSDs), Hard Disk Drives (HDDs), Non-Volatile Memory Express (NVMe) SSDs, and the like. The memory stores software like operating systems, MEG applications, localization applications, Proportional Integral Derivative (PID) control applications, sensor data, and the like. The processors retrieve and execute the software from the memory to drive the operation of controller 141.

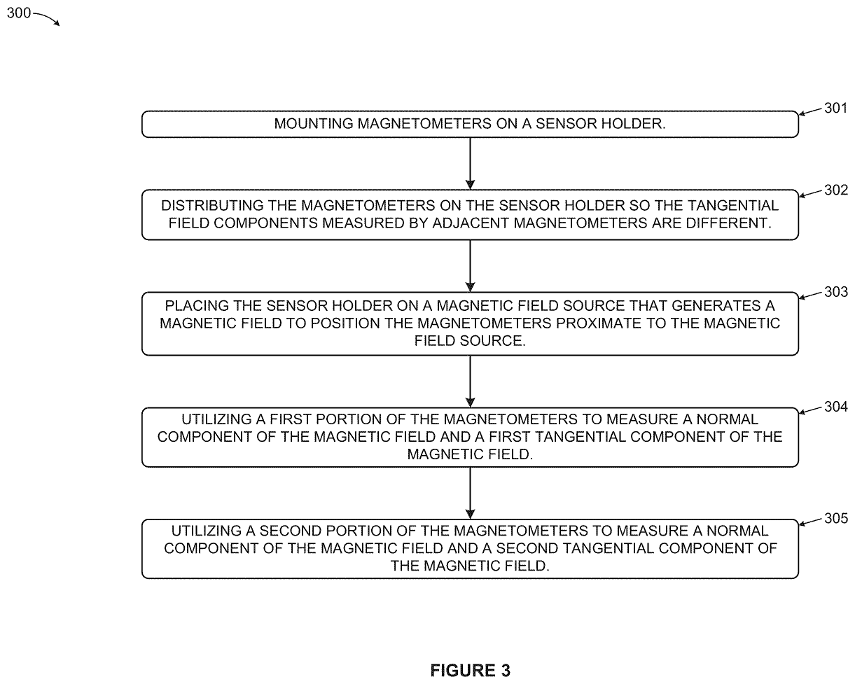

In some examples, magnetic field detection system 100 implements process 300 illustrated in FIG. 3. It should be appreciated that the structure and operation of system 100 may differ in other examples.

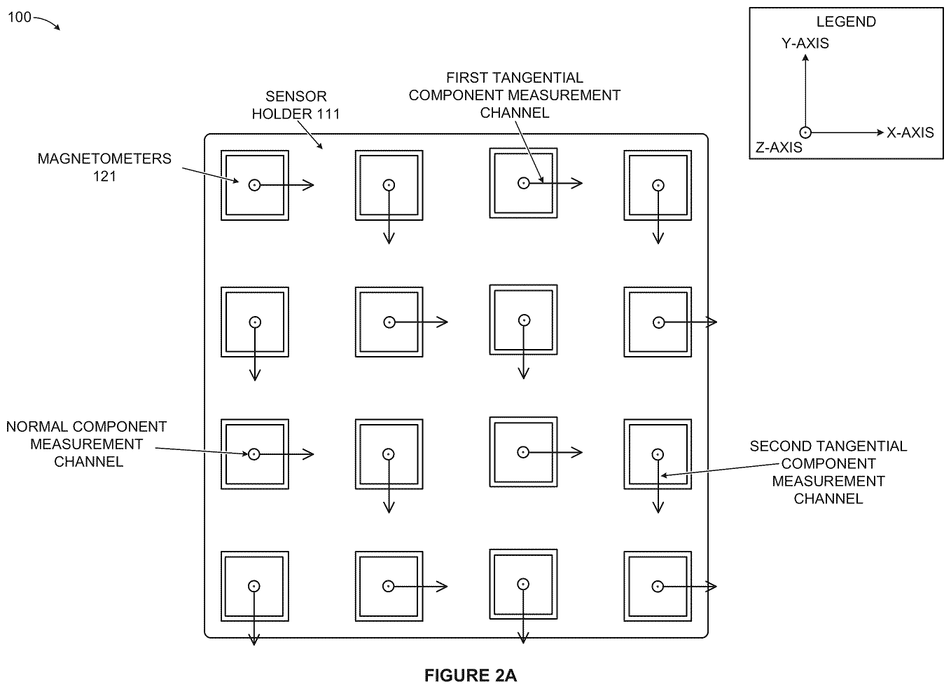

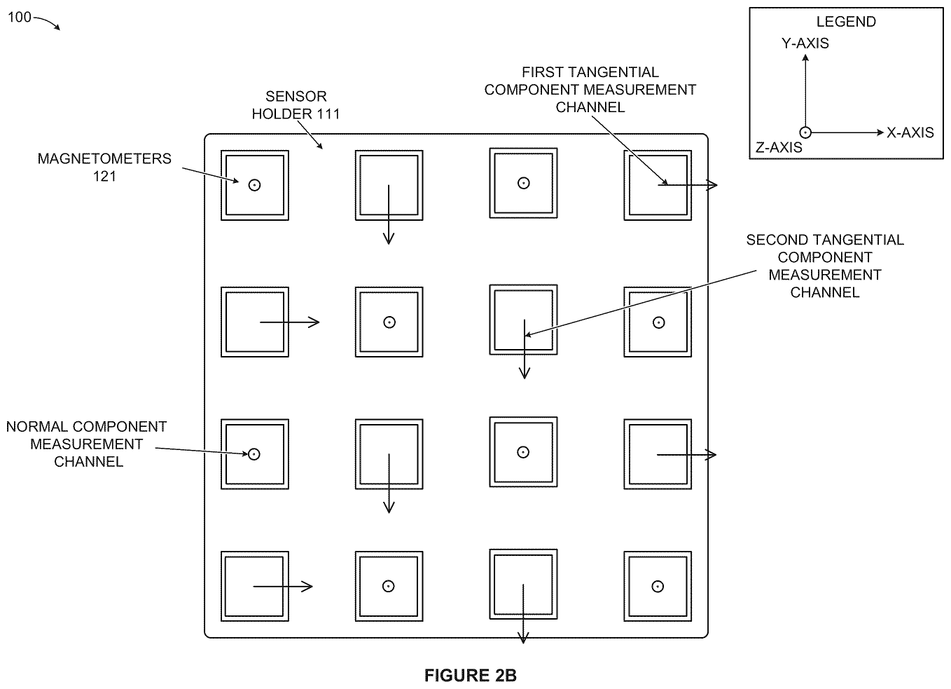

FIGS. 2A and 2B illustrate sensor layouts on sensor holder 111 for single-axis and dual-axis magnetometers. FIGS. 2A and 2B provide a top-down view of magnetic field detection system 100. As such, the view of magnetic field source 101 is obstructed by sensor holder 111 and magnetometers 121. Since FIG. 2A illustrates a top-down view, the z-axis now runs through the page, the y-axis runs along the vertical of the page, and the x-axis remains in the same orientation as illustrated in FIG. 1.

FIG. 2A illustrates a dual-axis sensor layout of magnetometers 121 on sensor holder 111 in magnetic field detection system 100. The slots of sensor holder 111 are organized into rows and columns. In some examples, magnetometers 121 comprise dual-axis magnetometers and each comprise a normal measurement channel and one of two tangential measurement channels to maintain the normal component as the dominant measuring mode. As illustrated in FIG. 2A, the tangential measurement channels of neighboring ones of magnetometers 121 are different to evenly distribute the tangential field measurements. In this example, neighboring magnetometers are ones of magnetometers 121 that reside in horizontally/vertically adjacent slots of sensor holder 111. However, it should be appreciated that the geometry of sensor holder 111 may be different in other examples and the definition of neighboring will change correspondingly. For example, sensor holder 111 may comprise a curved geometry (e.g., to fit around a human head), and neighboring ones of magnetometers 121 may reside in adjacent slots on the curved geometry.

FIG. 2B illustrates a single-axis sensor layout of magnetometers 121 on sensor holder 111 in magnetic field detection system 100. In some examples, magnetometers 121 comprise single-axis magnetometers and each comprise either a normal measurement channel or one of two tangential measurement channels. As illustrated in FIG. 2B, every other slot of holder 111 comprises a magnetometer with a normal component measurement channel to maintain the normal component as the dominant measuring mode. The remaining slots of holder 111 comprise magnetometers with one of the two tangential measurement channels. The first and second tangential component measurement channels alternate along the rows of sensor holder 111 to evenly distribute the tangential field measurements.

FIG. 3 illustrates process 300. Process 300 comprises an exemplary operation of magnetic field detection system 100 to arrange magnetometers. In other examples process 300 may differ. The operations of process 300 comprise mounting magnetometers on a sensor holder (step 301). The operations further comprise distributing the magnetometers on the sensor holder, so the tangential field components measured by adjacent magnetometers are different (step 302). The operations further comprise placing the sensor holder on a magnetic field source that generates a magnetic field to position the magnetometers proximate to the magnetic field source (step 303). The operations further comprise utilizing a first portion of the magnetometers to measure a normal component of the magnetic field and a first tangential component of the magnetic field (step 304). The operations further comprise utilizing a second portion of the magnetometers to measure the normal component of the magnetic field and a second tangential component of the magnetic field (step 305).

Referring back to FIG. 1, magnetic field detection system 100 includes a brief example of process 300 as implemented by the various components of system 100. In some examples, an operator inserts magnetometers 121 into sensor holder 111. Once inserted, sensor holder 111 secures magnetometers 121 (step 301). The operator orients magnetometers 121 in every other slot of sensor holder 121 to measure the normal magnetic field component and the first tangential magnetic field component. The operator orients the remaining ones of magnetometers 121 to measure the normal magnetic field component and the second tangential magnetic field component. By orienting magnetometers 121 in this way, sensor holder 111 distributes magnetometers 121 so the tangential field component measured by adjacent magnetometers are different. The operator places sensor holder 111 on magnetic field source 101 and presses magnetometers 121 through their respective slots until in contact with magnetic field source 101 (step 303). Once in contact, sensor holder 111 locks the orientation and position of magnetometers 121. Controller 141 receives a user input from the operator that initiates magnetic field sensing. Controller 141 generates control signaling that directs magnetometers 121 to sense the magnetic field and transfers the control signaling to magnetometers 121 over cabling 131. Magnetometers 121 operate in response to the control signaling. The ones of magnetometers 121 oriented to measure the normal and first tangential components measure the magnetic field and generate data characterizing field strength on the normal and first tangential axis (step 304). The ones of magnetometers 121 oriented to measure the normal and second tangential components measure the magnetic field and generate data characterizing field strength on the normal and second tangential axis (step 305). Magnetometers 121 transfer the data to controller 141. Controller 141 processes this data to generate an image depicting the sensed magnetic fields.

Advantageously, magnetic field detection system 100 efficiently allocates measurement resources. Moreover, magnetic field detection system 100 effectively distributes the normal and tangential measurement channels of magnetometers 121 to include both tangential measurement axes with respect to the surface of magnetic field source 101 while maintaining the measurement axis normal to the surface of magnetic field source 101 as the dominant measurement mode. By efficiently distributing single or dual axis magnetometers, magnetic field detection system 100 achieves similar magnetic imaging results to three-axis magnetometer systems at a reduced cost.

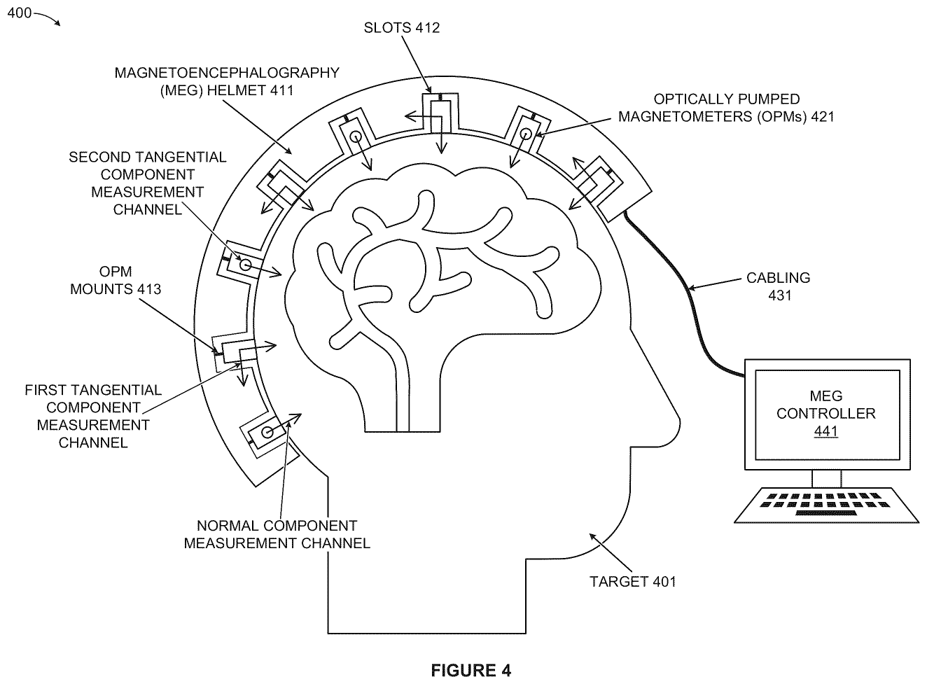

FIG. 4 illustrates MEG system 400. MEG system 400 performs operations like detecting anatomical magnetic fields and relating the detected magnetic fields to neuronal activity for use in medical applications. Exemplary medical applications include identifying brain activity and diagnosing medical conditions like stroke, epilepsy, arrhythmia, neuronal injuries, neuronal disorders, and/or other types of medical conditions relating to brain or neuron activity. MEG system 400 comprises an example of magnetic field detection system 100 illustrated in FIG. 1, however system 100 may differ. MEG system 400 comprises target 401, MEG helmet 411, OPMs 421, cabling 431, and MEG controller 441. MEG helmet 411 comprises slots 412 and OPM mounts 413. MEG helmet 411 may comprise additional components like localization coils, padding, chinstraps, support members, and the like however the additional components are omitted for clarity. As illustrated in FIG. 4, each of OPMs 421 comprises two measurement channels, a normal component measurement channel and one tangential component measurement channel. In other examples, each of OPMs 421 may comprise a single measurement channel, either a normal component measurement channel or a tangential component measurement channel. The normal component channel for each of OPMs 421 measures magnetic field components normal to the surface of target 401 while the tangential component channels for each of OPMs 421 measure magnetic field components that are not normal to the surface of target 401. For example, the first and second tangential measurement channels may be orthogonal or near orthogonal (e.g., within 10° of orthogonal) to the normal measurement channels and to each other. In other examples, MEG system 400 may differ. MEG system 400 may include fewer or additional components than those illustrated in FIG. 4. Likewise, the illustrated components of system 400 may include fewer or additional components, assets, or connections than shown.

In some examples, MEG helmet 411 comprises wearable headgear configured to position OPMs 421 in locations proximate to target 401. For example, MEG helmet 411 may securely adhere OPMs 421 to the scalp of target 401 using mechanical constraints to contour OPMs 421 to the surface of target 401. In this example, MEG helmet 411 comprises a rigid helmet and may be constructed from rigid plastic, carbon fiber, polymer, or other types of materials that provide structural support to MEG helmet 411 and that do not interfere in the magnetic sensing operations of OPMs 421. For example, MEG helmet 411 may comprise a 3D printed construction. OPMs 421 reside in slots 412. Slots 412 comprises indented regions (or through holes) in MEG helmet 411 shaped to house OPMs 421. For example, if OPMs 421 are rectangular prisms, slots 412 may comprise indentations shaped to correspond to the rectangular prism shape and size of OPMs 421. Slots 412 control the sensing direction of OPMs 421. For example, slots 412 may be shaped to orient the measurement channels of a first portion of OPMs 421 normally to the surface of target 401 and to orient the measurement channels of a second portion of OPMs 421 tangentially to the surface of target 401.

OPMs 421 are attached to MEG helmet 411 in slots 412 via OPM mounts 413 to control the position and orientation of OPMs 421. OPM mounts 413 may comprise clamps, ratchet mechanisms, set screws, springs, pistons, pneumatics, electric actuators, and the like. Slots 412 are shaped to constrain the three orientational and two of the locational degrees-of-freedom for each of OPMs 421. OPM mounts 413 control the last locational degree of freedom for each of OPMs 421 to move each of OPMs 421 through slots 412 along their respective axes of motion to desired locations. For example, set screws in the OPM mounts may be tightened to move OPMs 421 to contact target 401. For example, OPM mounts 413 may comprise actuators and electronic pistons. The actuators may receive control signaling and in response, drive the electronic pistons to move OPMs 421 through slots 412 until in contact with the scalp target 401. Once at the desired location (e.g., in contact with target 401), OPM mounts 413 lock to secure OPMs 421 at their desired locations. In alternate examples, OPMs 421 instead protrude from MEG helmet 411 and retract into slots 412 along their axes of motion when MEG helmet 411 is worn by target 401. For example, MEG helmet 411 may be placed onto the head of a target 401 and the head may force OPMs 421 into slots 412. In this case, the ratchet mechanism may comprise springs that allow OPMs 421 to compress into slots 412 in response to MEG helmet 411 being worn by target 401.

In alternate examples, MEG helmet 411 may comprise a flexible cap instead of a rigid helmet. The flexible cap may comprise an elastic material like rubber, clastic fabric, and the like. The flexible cap forms naturally to the shape of target 401 and compresses OPMs 421 onto the scalp of target 401 to fix in place both the position and orientation of OPMs 421. In the case where MEG helmet 411 comprises a flexible cap, slots 412 and ratchet mechanisms 113 may be replaced with different slots that orient the measurement channels of OPMs 421 along normal channels and tangential axes with respect to the surface of target 401.

OPMs 421 are atomic magnetometers to perform MEG. OPMs 421 comprise components like probe lasers, pump lasers, collimating lenses, quarter wave plates, vapor cells, photodetectors, compensating coils, bias coils, and heaters. OPMs 421 may include signal processors and other electronics. OPMs 421 may comprise single-axis or dual-axis magnetometers. A single-axis OPM may sense magnetic field components along a single measurement axis while a dual-axis OPM may sense magnetic field components along two measurement axes. In this example, OPMs 421 comprise dual-axis OPMs. As illustrated in FIG. 4, one of the measurement channels for each of OPMs 421 is aligned normally to the surface of target 401 while the other measurement channel is aligned tangentially to the surface of target 401 on a plane in line with the page or running through the page. The normal and tangential measurement channels for OPMs 421 are orthogonal or near orthogonal (e.g., within 10 degrees) to each other. The tangential measurement channels of OPMs 421 in adjacent ones of slots 412 are different. The direction of the measurement channels may be controlled physically via slots 412 and/or electrically via control signaling generated by MEG controller 441. OPMs 421 are contoured to the head of target 401 and sense magnetic fields generated by a neuronal activity in target 401 along their respective measurement channels. The neuronal activity in the brain of target 401 comprises intercellular electromagnetic signals. OPMs 421 sense the magnetic component of the electromagnetic signals to detect the neuronal activity over their measurement channels. OPMs 421 generate signals that characterize the strength, direction, and/or some other aspect of the detected magnetic fields.

OPMs 421 are coupled to MEG controller 441 over cabling 431. Cabling 431 comprises sheathed metallic wires. For example, OPMs 421 may transfer signaling that characterizes the sensed magnetic field to MEG controller 441 over cabling 431. Cabling 431 may be detachably coupled to MEG controller 441 and/or OPMs 421. In some examples, cabling 431 may be replaced with, or used in addition with, a wireless transceiver system (e.g., antennas) to transfer communications between MEG controller 441 and OPMs 421 over a wireless networking protocol like bluetooth.

MEG controller 441 is representative of one or more computing devices configured to drive the operation of OPMs 421 to generate magnetic based images (e.g., MEG images) that model neuronal activity in target 401. The one or more computing devices comprise processing circuitry, memories, lock-in amplifiers, modulators, PID controllers, power supply, displays, user components, and transceivers that are connected over bus circuitry. The processing circuitry may comprise CPUs, GPUs, DSPs, ASICs, FPGAs, and the like. The lock-in amplifiers comprise demodulators and low-pass filters. The memories may comprise RAM, flash circuitry, SSDs, HDDs, NVMe SSDs, and the like. The memory stores software like operating systems, MEG applications, localization applications, PID control applications, sensor data, and the like. The processors retrieve and execute the software from the memory to drive the operation of controller 441.

In operation, MEG helmet 411 is placed on the head of target 401. A human operator adjusts OPM mounts 413 to drive OPMs 421 through their respective ones of slots 412 until in contact with the scalp of target 401. OPM mounts 413 locks to constrain the positions and orientations of OPMs 421 and conform OPMs 421 to the shape of target 401. The human operator interacts with a user interface system of MEG controller 441 to initiate MEG imaging. MEG controller 441 receives the user input and in response, delivers current and control signaling to OPMs 421 over cabling 431. The current powers OPMs 421 and the control signaling directs OPMs to measure the magnetic field generated by target 401. MEG controller 441 applies modulation patterns to the current to orient the measurement channels of OPMs 421. The modulation patterns drive bias coils in OPMs 421 to generate bias magnetic fields that orient the sensing axes of OPMs 421 along the normal and tangential measurement channels as illustrated in FIG. 4.

OPMs 421 receive the current and control signaling over cabling 431. The vapor cells in OPMs 421 are positioned in the magnetic field of target 401. The bias coils in OPMs 421 receive the current and generate bias fields that orient the measurement channels of OPMs 421 based on the modulation patterns. The heaters receive the current and heat the vapor cells and lasers in OPMs 421. The lasers emit a beam that is circularly polarized at a resonant frequency of the vapor to polarize the atoms in the vapor cells. The beams pass through the collimating lenses, quarter wave plates, and enter the vapor cells polarizing and probing the vapor. The presence of the magnetic field alters the optical properties of the vapor, which in turn are mapped onto the transmitted light beam. Changes in the optical properties correlate to the magnetic field. The photodetectors detect the transmitted light beam by transducing changes in its optical properties into electronic signals. The photodetectors generate and transfer corresponding analog electronic signals that characterize the magnetic field along the normal and tangential measurement channels to MEG controller 441 over cabling 431.

MEG controller 441 receives the signaling that indicates the measured magnetic field strength. MEG controller 441 executes imaging applications that perform source localization to generate a magnetic field image. MEG controller 441 filters out noise artifacts based on magnetic field strengths captured by the tangential measurement channels of OPMs 441. MEG controller 441 generates the image based on the target magnetic field strengths measured by the normal channels of OPMs 421, the spatial locations for each of OPMs 421, and the orientational/positional constraints on OPMs 421. The images depict the magnetic field detected by OPMs 421 in three dimensions to model the neuronal activity in the brain of target 401.

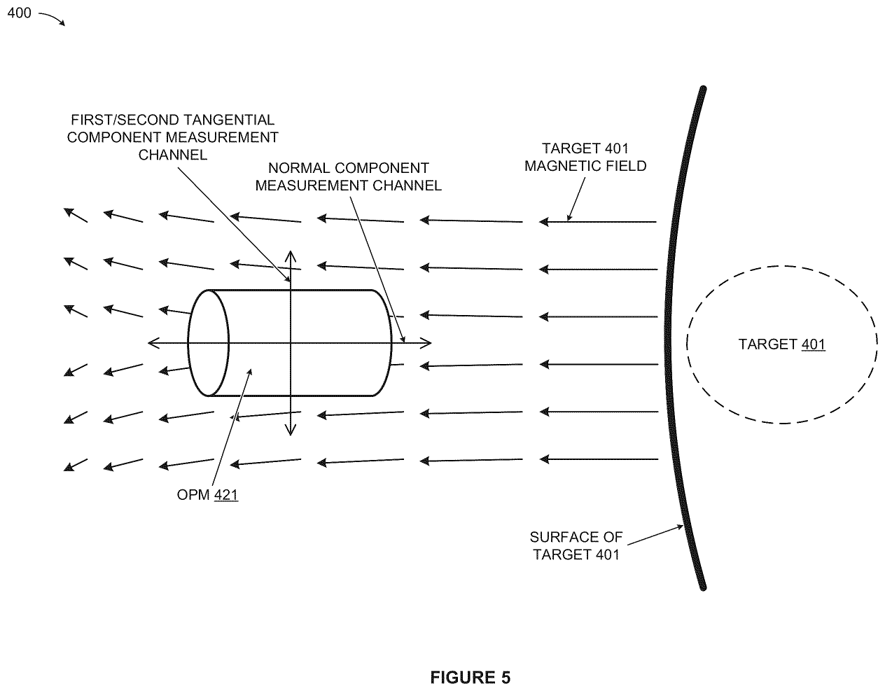

FIG. 5 further illustrates OPMs 421 in MEG system 400. OPMs 421 are referred to in the singular for sake of clarity. FIG. 5 illustrates the tangential and normal component measurement channels of OPM 421 in a magnetic field generated by target 401. The magnetic field of target 401 is illustrated as arrows. The arrows represent the field vectors that comprise the magnetic field. The direction of the arrows indicates the direction of the magnetic field at a given spatial location. The length of the arrows indicates the magnitude of magnetic field at a given spatial location. OPM 421 is positioned in the magnetic field. OPM 421 detects magnetic fields components along its measurement channels. Specifically, OPM 421 may detect field components aligned with its measurement channels. As the measurement channels of OPM 421 change with respect to the magnetic field, OPM 421 detects different field components of the magnetic field. In this example, OPM 421 is positioned to detect magnetic field components normal to and tangential to the surface of target 401. In some examples, OPM 421 comprises a single measurement channel.

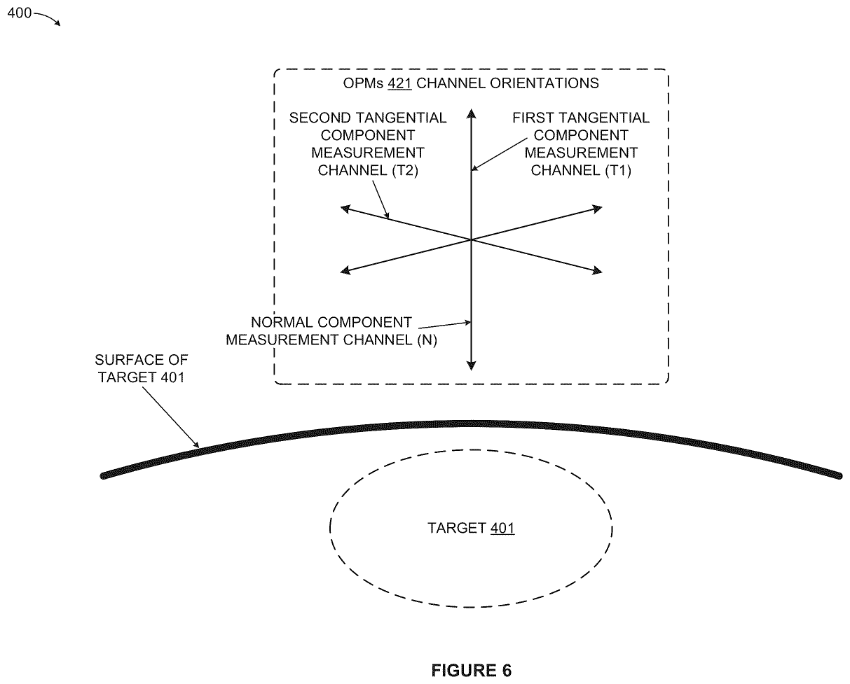

FIG. 6 illustrates the measurement channel orientations of OPMs 421 in MEG system 401. OPM 421 channel directions comprise a normal component measurement channel (N), a first tangential measurement channel (T1), and a second tangential measurement channel (T2). The channel orientations are representative of sensor orientations to measure the magnetic field generated by target 401 as well as ambient magnetic noise. The normal component measurement channel is normal to the surface of target 401. In this example, the two tangential components comprise measurement directions orthogonal to the normal component and to each other. As such, all three measurement components form 90° angles between each other. In other examples, one or more of the tangential components may comprise other orientations (e.g., non-orthogonal) than those illustrated in FIG. 6.

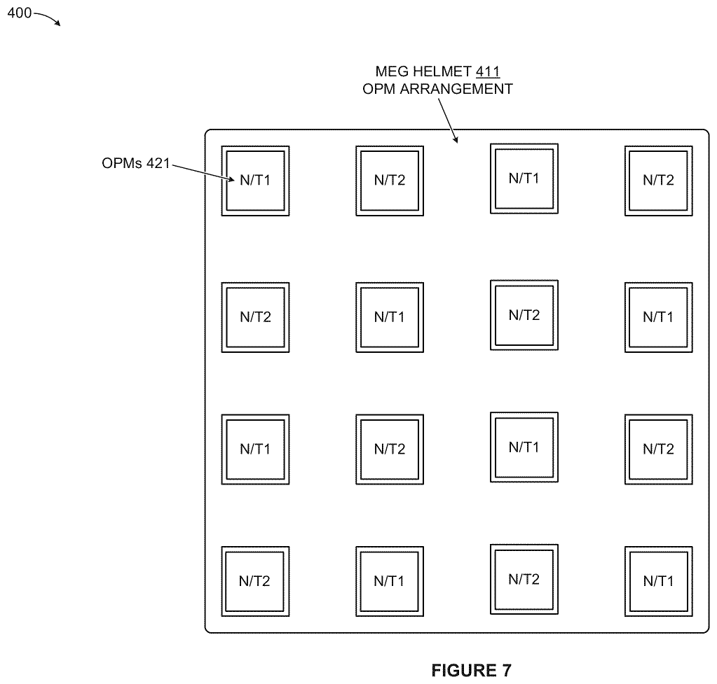

FIG. 7 illustrates an exemplary OPM arrangement for MEG helmet 411 in MEG system 400 for dual-axis OPMs. Each of OPMs 421 comprises a normal measurement direction (N) and one of two tangential measurement directions (T1, T2) that are orthogonal to the normal direction as depicted in FIG. 6. The OPM arrangement of MEG helmet 411 comprises a measurement orientation map for a set of dual-axis OPMs mounted to MEG helmet 411. The arrangement indicates two channel directions for OPMs 421 for each of slots 412. In each of slots 412, one of OPMs 421 is oriented to measure the normal component of the magnetic field of target 401 and one of the two tangential directions. In doing so the normal magnetic field remains the preferred measurement channel for target 401's magnetic field. As illustrated in FIG. 7, horizontally/vertically adjacent ones of slots 412 alternate between the two tangential channel directions to distribute tangential measurements across MEG helmet 411.

Advantageously, the OPM arrangement of MEG helmet 411 efficiently allocates magnetic field measurement resources in MEG system 400. Moreover, the arrangement effectively optimizes the geometry of sensor components which reduces the needed resources to reconstruct the target magnetic fields and reject ambient magnetic field noise. The efficient distribution of two-channel magnetometers allows for the equivalent measurement fidelity to three-channel magnetometer arrangements at a lower cost.

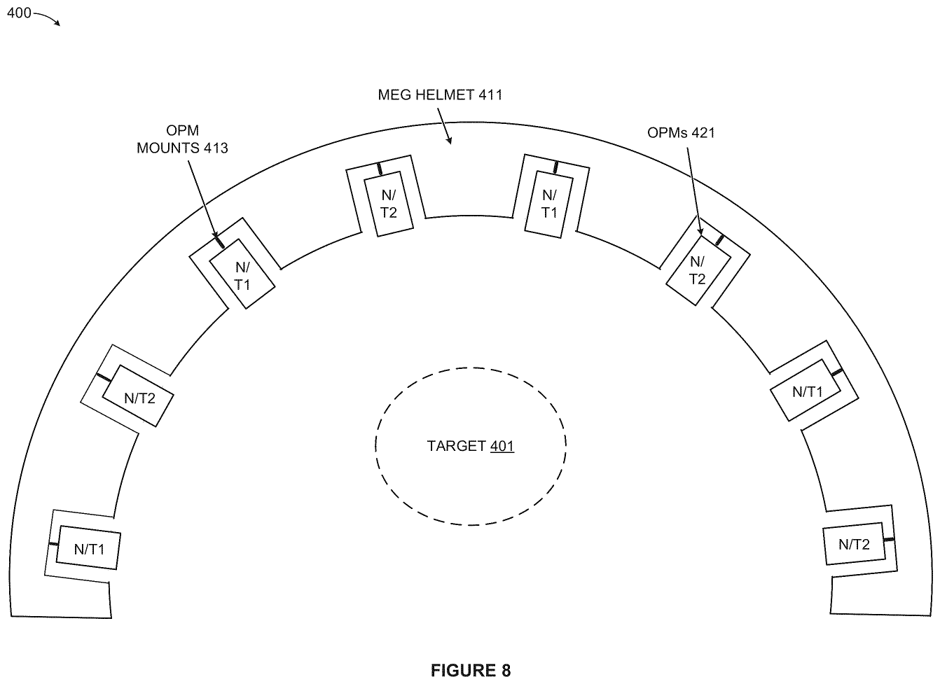

FIG. 8 further illustrates MEG helmet 411 utilizing the OPM sensor arrangement illustrated in FIG. 7 in MEG system 400. MEG helmet 411 comprises an example of sensor holder 111 illustrated in FIGS. 1 and 2, however holder 111 may differ. In some examples, OPMs 421 comprise two-channel OPMs that can measure magnetic field strength along two measurement channels. Slots 412 orient OPMs 621 to algin one of their measurement channels with a normal component of a magnetic field and the other measurement channel with one of two tangential components of a magnetic field. As illustrated in FIG. 8, adjacent ones of OPMs 421 alternate between the two tangential components to evenly distribute tangential field measurements across MEG helmet 411 while maintaining the normal component as the dominant measurement mode. For example, every other one of OPMs 421 may be rotated 90° to measure the other tangential field component. Alternatively, MEG controller 441 may vary modulation patterns in the current delivered to the bias coils in OPMs 421 to enable the alternating tangential measurement channels as depicted in FIG. 8. Generally, the number of normal measurements channels is equal to or greater than the total number of tangential measurement channels. Alternatively, the number of normal measurement channels may constitute a plurality of the total number of measurement channels.

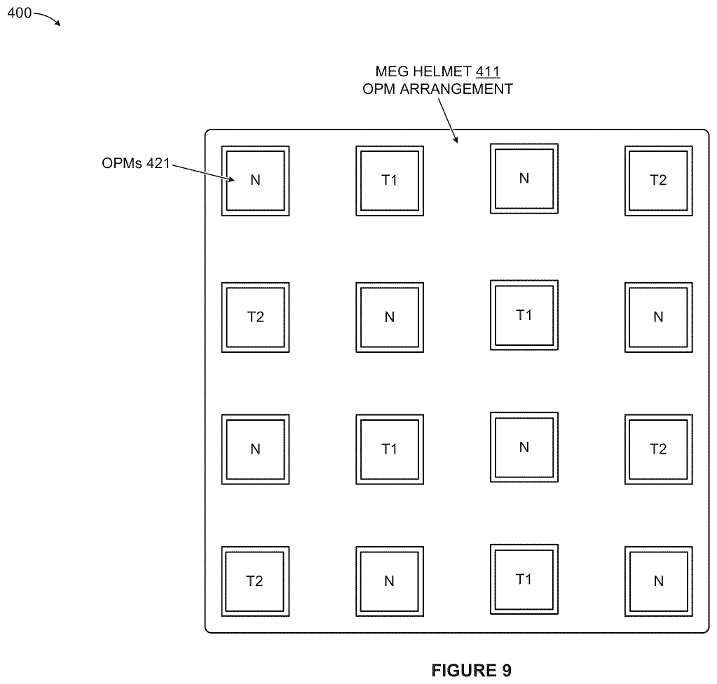

FIG. 9 illustrates an exemplary OPM arrangement for MEG helmet 411 in MEG system 400 for single-axis OPMs. Each of OPMs 421 comprises one of a normal measurement direction (N) and one of two tangential measurement directions (T1, T2) that are orthogonal to the normal direction as depicted in FIG. 6. The OPM arrangement of MEG helmet 411 comprises a measurement orientation map for a set of single-axis OPMs mounted to MEG helmet 411. The arrangement indicates one channel direction for OPMs 421 for each of slots 412. In each of slots 412, OPMs 421 are either oriented to measure the normal component of the magnetic field of target 401 or one of the two tangential directions. As illustrated in FIG. 9, adjacent ones of OPMs 421 alternate between the two tangential directions and the normal direction to distribute tangential measurements and maintain the normal direction as the dominant measurement mode.

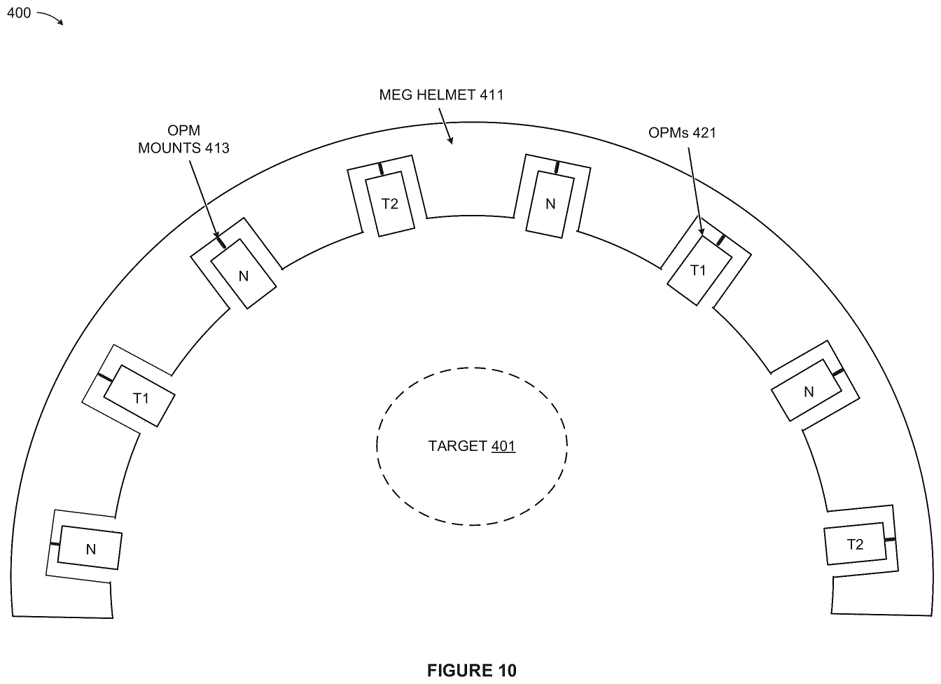

FIG. 10 further illustrates MEG helmet 411 utilizing the OPM sensor arrangement illustrated in FIG. 9 in MEG system 400. In some examples, OPMs 421 comprise single-channel OPMs that can measure magnetic field strength along one measurement channel. Slots 412 orient a first portion of OPMs 421 to algin their measurement channels with a normal component of a magnetic field, orient a second portion of OPMs 421 to algin their measurement channels with a first tangential component of the magnetic field, and orient a third portion of OPMs 421 to algin their measurement channels with a second tangential component of the magnetic field. As illustrated in FIG. 10, adjacent ones of OPMs 421 alternate between normally orientated measurement channels and one of the two tangential orientated measurement channels to evenly distribute tangential field measurements across MEG helmet 411 while maintaining the normal component as the dominant measurement mode. For example, the channel direction of the normally oriented OPMs may be aligned towards the surface of target 401, the channel direction of the first tangentially oriented OPMs may be aligned parallel to the surface of target 401 in line with the page, and the second tangentially oriented OPMs may be aligned parallel to the surface of target 401 directionally through the page. OPMs 421 may be physically rotated to control their measurement channel directions. Alternatively, MEG controller 441 may vary modulation patterns in the current delivered to the bias coils in OPMs 421 to enable the alternating tangential measurement channels as depicted in FIG. 10.

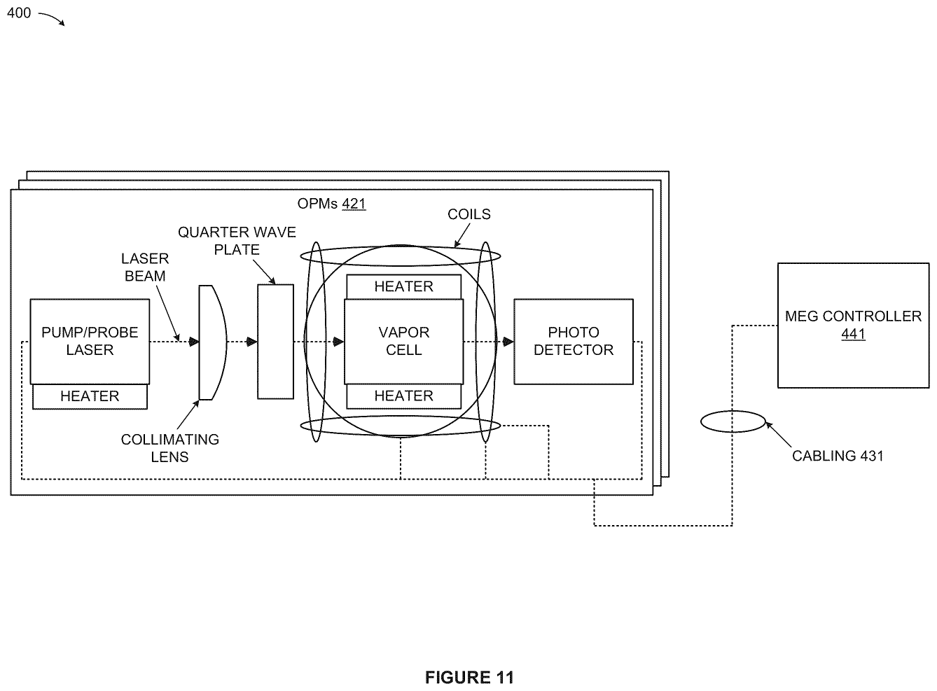

FIG. 11 further illustrates OPMs 421. OPMs 421 comprises an example of magnetometers 121 illustrated in FIGS. 1, 2A, and 2B, however magnetometers 121 may differ. OPMs 421 are referred to in the singular for sake of clarity. OPM 421 is representative of an intrinsically dual-axis single-beam magnetometer (e.g., sensitive to magnetic fields on both orthogonal axes with respect to the light beam propagation axis) or an intrinsically single-axis single-beam magnetometer (e.g., sensitive to magnetic fields on one orthogonal axis with respect to the light beam propagation axis). OPM 421 comprises a pump/probe laser, collimating lens, quarter wave plate, vapor cell, photodetector, heaters, and coils.

In some examples, MEG controller 441 routes control signaling and/or current to the laser, photodetector, heaters, and coils over cabling 431 and bus circuitry within OPM 421. MEG controller 441 includes a modulation pattern in the current supplied to the coils to control the magnetic field produced by the coils. The coils are positioned around the vapor cell and comprise loops of metallic wiring within OPM 421 that generate an electromagnetic field in response to receiving electric current. Coils emit a compensation magnetic field to null background magnetic fields and/or emit a bias magnetic field to orient the sensing direction of OPM 421 depending on the modulation pattern in the current received from MEG controller 441. The heaters comprise resistive heat elements operatively coupled to the pump/probe laser and the vapor cell. The heaters heat pump/probe laser to facilitate beam generation and heat the vapor cell to increase cell pressure to facilitate magnetic field sensing.

The pump/probe laser comprises a light source to optically pump and probe the metallic vapor housed by the vapor cell. The laser produces a single laser beam in response to power/signaling received from MEG controller 441. The collimating lens comprises a glass piece that focuses the laser beam along a single propagation axis. The quarter wave plate is an optical device that alters the polarization state of the laser beam. The vapor cell comprises an atomic device that encloses metallic vapor. The vapor cell is positioned within the magnetic field generated by neuronal activity in target 401. The vapor cell may comprise a glass vapor cell, a silicon-glass vapor cell, and/or another type of vapor cell. The metallic vapor is selected for comprising properties relating to magnetic field sensitivity. Typically, the metallic vapor comprises an alkali metal vapor like rubidium vapor, cesium vapor, or potassium vapor. The vapor cell may enclose buffer gases (e.g., nitrogen or helium) in addition to the metallic vapor. The photodetector comprises a light sensor to absorb and measure the intensity of the beam emitted by pump/probe laser. When the probe beam passes through the vapor cell, a portion of the beam is absorbed by the metallic vapor. The remaining portion of the beam exits the cell and is detected by the photodetector. The amount of light absorbed by the metallic vapor correlates to the components of the external magnetic field at the location of vapor cell. The photodetector routes magnetic field measurement signals to MEG controller 441 over the bus circuitry of OPM 421 and cabling 431.

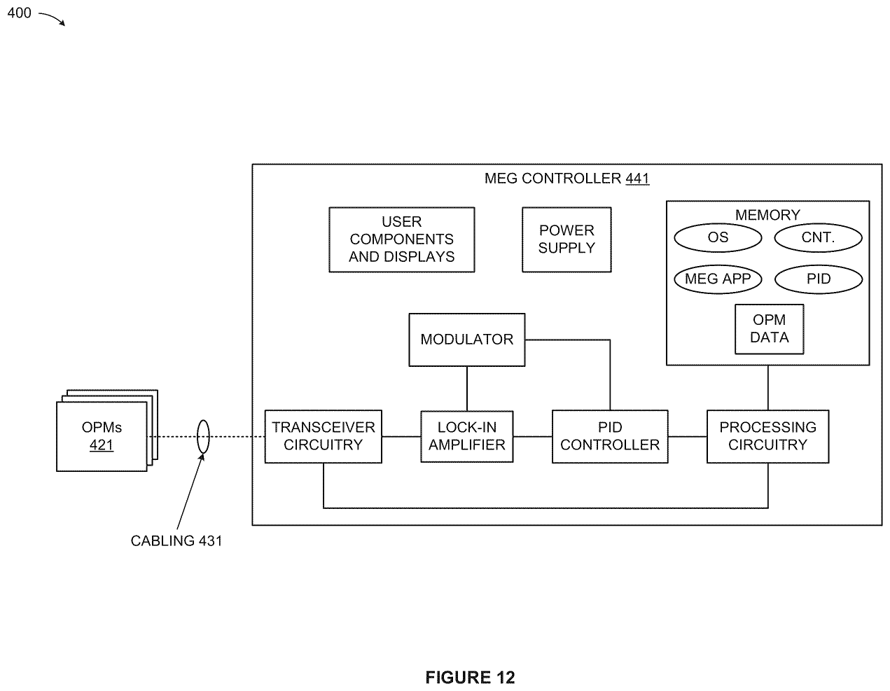

FIG. 12 further illustrates MEG controller 441 in MEG system 401. MEG controller 441 comprises an example of controller 141 illustrated in FIG. 1, however controller 141 may differ. MEG controller 441 comprises transceiver circuitry (XCVR), memory, processing circuitry, a PID controller, a lock-in amplifier, a modulator, power supply, and user components and displays connected over bus circuitry. The processing circuitry comprises a CPU, GPU, DSP, FPGA, ASIC, and/or some other type of processing circuitry. The lock-in amplifier comprises a low-pass filter and demodulator. The memory comprises RAM, HDD, SSD, NVMe SSD, and the like. The memory stores an operating system, control application, MEG application, PID application, and OPM data. The processing circuitry retrieves the software from the memory and executes the software to drive the operation of MEG system 400 as described herein.

In some examples, the operating system manages the hardware and software resources of MEG controller 441. The control application controls the measurement operations of OPMs 421. The MEG application generates MEG images based on the magnetic field measurements reported by OPMs 421. The sensor data comprises operating parameters of OPMs 421 like sensor Identifier (ID), slot ID, configuration parameters, and sensor performance metrics. The PID application drives the PID controller to generate control outputs to tune the fields produced by the coils in OPMs 421. The control outputs may be used to read out the vector components of target 401's magnetic field. The transceiver circuitry passes photodetector signals to the lock-in amplifier. The lock-in amplifier demodulates the signals received from OPMs 421 and passes demodulated photodetector signals below a certain frequency to the PID controller. The modulator generates a reference frequency/phase for the lock-in amplifier. The PID controller processes output from the low-pass filter to generate control signaling to tune the fields generated by the coils. The modulator generates modulation patterns to control the sensing direction of OPMs 421.

The processing circuitry controls OPMs 421 to sense components of the magnetic field (e.g., magnetic fields generated by target 451) along one or two measurement directions. In particular, the processing circuitry applies the modulation pattern generated by the modulator and the control output generated by the PID controller into the currents supplied to coils in OPMs 421 to allow for magnetic field components at the location of the vapor cells to be derived along all normal and/or tangential measurement axis. For example, when implementing the OPM arrangement illustrated in FIG. 7, MEG controller 441 may apply modulation patterns to the current delivered to the coils in OPMs 421 to orient their measurement channels as depicted in FIG. 8. For example, when implementing the OPM arrangement illustrated in FIG. 9, MEG controller 441 may apply modulation patterns to the current delivered to the coils in OPMs 421 to orient their measurement channels as depicted in FIG. 10. The processing circuitry delivers the current, control outputs, and modulation patterns to OPMs 421 to drive the operation of OPMs 421. The processing circuitry processes the control outputs generated by the PID controller to estimate magnetic field components along the normal and/or tangential measurement axes of OPMs 421.

The above Technical Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having operations, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Technical Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Technical Description section explicitly defines such terms. Accordingly, the actual scope of the technology encompasses not only the disclosed examples, but also all equivalent ways of practicing or implementing the technology under the claims.

What is claimed is:

1. A magnetic field detection system to arrange magnetometers, the magnetic field detection system comprising:
a sensor holder configured to mount the magnetometers proximate to a magnetic field source that generates a magnetic field;
the magnetometers configured to measure the magnetic field in multiple directions, wherein a first portion of the magnetometers measures a normal component of the magnetic field and a first tangential component of the magnetic field, and a second portion of the magnetometers measures the normal component of the magnetic field and a second tangential component of the magnetic field; and
the sensor holder configured to distribute the first portion of the magnetometers and the second portion of the magnetometers, so the tangential components measured by neighboring ones of the magnetometers mounted to the sensor holder are different.

2. The system of claim 1 wherein the first portion of the magnetometers and the second portion of the magnetometers are evenly distributed on the sensor holder.

3. The system of claim 1 wherein the tangential components measured by the neighboring ones of the magnetometers mounted to the sensor holder are orthogonal.

4. The system of claim 1 wherein the tangential components measured by the neighboring ones of the magnetometers mounted to the sensor holder are within 10 degrees of orthogonal.

5. The system of claim 1 wherein the magnetometers comprise atomic magnetometers.

6. The system of claim 1 wherein the magnetometers comprise Optically-Pumped Magnetometers (OPMs).

7. The system of claim 1 wherein the magnetometers comprise gradiometers.

8. The system of claim 1 wherein the magnetometers comprise nitrogen vacancy centers.

9. The system of claim 1 wherein the magnetometers comprise Superconducting Quantum Interference Devices (SQUIDs).

10. The system of claim 1 wherein the sensor holder comprises a Magnetoencephalography (MEG) sensor holder.

11. The system of claim 1 wherein the sensor holder comprises a Magnetocardiography (MCG) sensor holder.

12. The system of claim 1 wherein the sensor holder comprises a Magnetomyography (MMG) sensor holder.

13. The system of claim 1 wherein the sensor holder comprises a Magnetogastrography (MGG) sensor holder.

14. The system of claim 1 wherein the sensor holder comprises a Magnetoneurography (MNG) sensor holder.

15. The system of claim 1 wherein the sensor holder comprises a helmet.

16. The system of claim 1 wherein the sensor holder comprises a flexible cap.

17. The system of claim 1 wherein the sensor holder comprises a sleeve.

18. The system of claim 1 wherein the sensor holder comprises a vest.

19. The system of claim 1 wherein the sensor holder comprises a blanket.

20. A method to arrange magnetometers, the method comprising:
mounting magnetometers on a sensor holder;
distributing the magnetometers on the sensor holder so tangential field components measured by adjacent ones of the magnetometers are different;
placing the sensor holder on a magnetic field source that generates a magnetic field to position the magnetometers proximate to the magnetic field source;
utilizing a first portion of the magnetometers to measure a normal component of the magnetic field and a first tangential component of the magnetic field; and
utilizing a second portion of the magnetometers to measure the normal component of the magnetic field and a second tangential component of the magnetic field.
